SEQUENCE LISTING (8 sequences)

SEQ ID NO: 1 - protein, 5 aa, Artificial
  Synthetic H-CDR1 (Rituximab)
SYNMH

SEQ ID NO: 2 - protein, 17 aa, Artificial
  Synthetic H-CDR2 (rituximab)
AIYPGNGDTSYNQKFKG

SEQ ID NO: 3 - protein, 12 aa, Artificial
  Synthetic H-CDR3 (Rituximab)
STYYGGDWYFNV

SEQ ID NO: 4 - protein, 10 aa, Artificial
  Synthetic L-CDR1 (Rituximab)
RASSSVSYIH

SEQ ID NO: 5 - protein, 7 aa, Artificial
  Synthetic L-CDR2 (Rtixumab)
ATSNLAS

SEQ ID NO: 6 - protein, 9 aa, Artificial
  Synthetic L-CDR3 (Rituximab)
QQWTSNPPT

SEQ ID NO: 7 - protein, 451 aa, Artificial
  Synthetic Heavy Chain of Rituximab
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 213 aa, Artificial
  Synthetic Light chain of Rituximab
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0270862 A1
(43) Pub. Date: Aug. 15, 2024

(54) USE OF B CELL DEPLETING AGENTS FOR THE TREATMENT OF RHEUMATIC HEART DISEASE

(71) Applicants: INSERM (INSTITUT NATIONAL DE LA SANTÉ ET DE LA RECHERCHE MÉDICALE), Paris (FR); UNIVERSITÉ PARIS CITÉ, Paris (FR); ASSISTANCE PUBLIQUE-HÔPITAUX DE PARIS (APHP), Paris (FR)

(72) Inventors: Xavier JOUVEN, PARIS (FR); Alexandre LOUPY, PARIS (FR)

(21) Appl. No.: 18/566,273

(22) PCT Filed: May 30, 2022
(86) PCT No.: PCT/EP2022/064616

§ 371 Date: Dec. 1, 2023

(30) Foreign Application Priority Data
Jun. 1, 2021 (EP) 21305727.6

(51) Int. Cl. C07K 16/28; A61K 39/00

(52) U.S. Cl. CPC C07K 16/2887; A61K 2039/505

(57) ABSTRACT

Rheumatic heart disease (RHD) ranks as one of the most serious cardiovascular scourges of the past century and remains a force to be reckoned with in the developing world. Using the B-HOT panel, developed in part by our team, and based on heatmap analysis, we show two distinct transcriptomic profiles between inflammatory burden region of RHD valves patients (n=13) and control valves (without lesions, n=10). Our differential gene expression analysis between those 2 groups show several genes overexpressed in RHD group compared to control valves. Interestingly, and as potential therapeutic targets, genes related to CD20 (MS4A1. MS4A2) are among genes that are differentially expressed. Taken together, these results suggest a potential role for CD20 in inflammatory responses of RHD valves lesions. Anti-CD20 antibodies, like Rituximab (CD20), would be potential therapeutic molecules to be used on RHD patients.

### FIELD OF THE INVENTION

The present invention is in the field of medicine, in particular cardiology.

### BACKGROUND OF THE INVENTION

Acute rheumatic fever (ARF) is a disease that can affect the heart, joints, brain, and skin.

Rheumatic fever can develop if streptococcus (Group A Streptococcus-GAS) infections are not treated properly. ARF is diagnosed by the updated Jones criteria which were first published in 1944. According to the updated Jones criteria, a diagnosis of ARF can be made when two major criteria (migratory polyarthritis; carditis; subcutaneous nodules; erythema marginatum; Sydenham's chorea), or one major criterion plus two minor criteria (fever; arthralgia; raised erythocyte sedimentation rate or C reactive protein; leukocytosis; ECG showing features of heart block) are present, along with evidence of GAS infection. The major clinically significant sequela of ARF is rheumatic heart disease (RHD) (Marijon E, Mirabel M, Celermajer D S, Jouven X. Rheumatic heart disease. Lancet. 2012 Mar. 10; 379(9819): 953-964). RHD can lead to serious cardiac involvement, with myocarditis or valvulitis leading to death or valve replacement. Systematic screening with echocardiography, as compared with clinical screening, reveals a much higher prevalence of rheumatic heart disease (Marijon E, Ou P, Celermajer D S, Ferreira B, Mocumbi A O, Jani D, Paquet C, Jacob S, Sidi D, Jouven X. Prevalence of rheumatic heart disease detected by echocardiographic screening. N Engl J Med. 2007 Aug. 2; 357(5):470-6). Throughout the developing world, RHD remains the leading cause of acquired heart disease in individuals younger than 50 years old.

### SUMMARY OF THE INVENTION

The present invention is defined by the claims. In particular, the present invention relates to use of B-cell depleting agents for the treatment of rheumatic heart disease.

### DETAILED DESCRIPTION OF THE INVENTION

The first object of the present invention relates to a method of treating rheumatic heart disease in a patient in need thereof comprising administering to the patient a therapeutically effective amount of a B-cell depleting agent.

As used herein, the term “rheumatic heart disease” or “RHD” has its general meaning in the art and refers to a group of short-term (acute) and long-term (chronic) heart disorders that are caused by rheumatic fever. While rheumatic heart disease (RHD) may develop after a single bout of acute rheumatic fever (ARF), it is typically associated with recurrent episodes of ARF. ARF usually occurs during childhood between the ages of 5 and 15 years. RHD can damage any part of the heart including the valves, the lining of the heart or the heart muscle, but more often damages the heart valves, especially those on the left side of the heart.

In some embodiments, the patient can be male or female. A patient can be one who has been previously diagnosed as having some symptoms of rheumatic heart disease. For example, a patient can be one who exhibits one or more risk factors for coagulation related disorder, or a patient who does not exhibit risk factors, or a patient who is asymptomatic for rheumatic heart disease. In some embodiments, the patient is younger than 65 years, preferably younger than 50 years old, more preferably younger than 40 years old, even more preferably younger than 25 years old.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “B cell depleting agent” refers to any agent that is capable of triggering lymphodepletion of B cells.

In some embodiments, the B cell depleting agent is an antibody having specificity for CD20.

As used herein, the term “CD20” has its general meaning in the art and refers to the B-lymphocyte antigen CD20 that is an activated-glycosylated phosphoprotein expressed on the surface of all B-cells beginning at the pro-B phase (CD45R+, CD117+) and progressively increasing in concentration until maturity. Human CD20 has the amino acid sequence of UniProt P011836.

As used herein the term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The light and heavy chains of an immunoglobulin each have three complementarity determining regions (CDRs), designated L-CDR1, L-CDR2, L-CDR3 and H-CDR1, H-CDR2, H-CDR3, respectively. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

In some embodiments, the antibody of the present invention is a chimeric antibody, typically a chimeric mouse/human antibody. The term “chimeric antibody” refers to a monoclonal antibody which comprises a VH domain and a VL domain of an antibody derived from a non-human animal, a CH domain and a CL domain of a human antibody. As the non-human animal, any animal such as mouse, rat, hamster, rabbit or the like can be used.

In some embodiments, the antibody is a humanized antibody. As used herein, the term “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the Fc region is modified to increase the ability of the antibody to mediate antibody dependent cellular cytotoxicity (ADCC) and/or to increase the affinity of the antibody for an Fc receptor by modifying one or more amino acids. In this manner the capabilities of the antibody to deplete B cells can be increased. This approach is described further in PCT Publication WO 00/42072 by Presta. Moreover, the binding sites on human IgGI for FcγRI, FcγRII, FcγRIII and FcRn have been mapped and variants with improved binding have been described (see Shields, R. L. et al, 2001 J. Biol. Chen. 276:6591-6604, WO2010106180). In some embodiments, the glycosylation of an antibody is modified. For example, an aglycoslated antibody can be made (i.e., the antibody lacks glycosylation).

Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies.

Examples of antibodies having specificity for CD20 include: “C2B8” which is now called “Rituximab” (“RITUXAN®”) (U.S. Pat. No. 5,736,137, expressly incorporated herein by reference), a chimaeric pan-B antibody targeting CD20; the yttrium-[90]-labeled 2B8 murine antibody designated “Y2B8” or “Ibritumomab Tiuxetan” ZEVALIN® (U.S. Pat. No. 5,736,137, expressly incorporated herein by reference), a murine IgGI kappa mAb covalently linked to MX-DTPA for chelating to yttrium-[90]; murine IgG2a “B1,” also called “Tositumomab,” optionally labeled with radioactive 1311 to generate the “1311-B1” antibody (iodine 131 tositumomab, BEXXAR™) (U.S. Pat. No. 5,595,721, expressly incorporated herein by reference); murine monoclonal antibody “IF5” (Press et al. Blood 69 (2):584-591 (1987) and variants thereof including “framework patched” or humanized 1F5 (WO03/002607, Leung, S.; ATCC deposit HB-96450); murine 2H7 and chimeric 2H7 antibody (U.S. Pat. No. 5,677,180, expressly incorporated herein by reference); humanized 2H7, also known as ocrelizumab (PRO-70769); Ofatumumab (Arzerra), a fully human IgGI against a novel epitope on CD20 huMax-CD20 (Genmab, Denmark; WO2004/035607 (U.S. Ser. No. 10/687,799, expressly incorporated herein by reference)); AME-133 (ocaratuzumab; Applied Molecular Evolution), a a fully-humanized and optimized IgGI mAb against CD20; A20 antibody or variants thereof such as chimeric or humanized A20 antibody (cA20, hA20, respectively) (U.S. Ser. No. 10/366,709, expressly incorporated herein by reference, Immunomedics); and monoclonal antibodies L27, G28-2, 93-1B3, B—CI or NU-B2 available from the International Leukocyte Typing Workshop (Valentine et al, In: Leukocyte Typing III (McMichael, Ed., p. 440, Oxford University Press (1987)). Further, suitable antibodies include e.g. antibody GA101 (obinutuzumab), a third generation humanized anti-CD20-antibody of Biogen Idec/Genentech/Roche. Moreover, BLX-301 of Biolex Therapeutics, a humanized anti CD20 with optimized glycosylation or Veltuzumab (hA20), a 2nd-generation humanized antibody specific for CD20 of Immunomedics or DXL625, derivatives of veltuzumab, such as the bispecific hexavalent antibodies of IBC Pharmaceuticals (Immunomedics) which are comprised of a divalent anti-CD20 IgG of veltuzumab and a pair of stabilized dimers of Fab derived from milatuzumab, an anti-CD20 mAb enhanced with InNexus' Dynamic Cross Linking technology, of Inexus Biotechnology both are humanized anti-CD20 antibodies are suitable. Further suitable antibodies are BM-ca (a humanized antibody specific for CD20 (Int J. Oncol. 2011 February; 38(2):335-44)), C2H7 (a chimeric antibody specific for CD20 (Mol Immunol. 2008 May; 45(10):2861-8)), PRO131921 (a third generation antibody specific for CD20 developed by Genentech), Reditux (a biosimilar version of rituximab developed by Dr Reddy's), PBO-326 (a biosimilar version of rituximab developed by Probiomed), a biosimilar version of rituximab developed by Zenotech, TL-011 (a biosimilar version of rituximab developed by Teva), CMAB304 (a biosimilar version of rituximab developed by Shanghai CP Guojian), GP-2013 (a biosimilar version of rituximab developed by Sandoz (Novartis)), SAIT-101 (a biosimilar version of rituximab developed by Samsung BioLogics), a biosimilar version of rituximab developed by Intas Biopharmaceuticals, CT-P10), a biosimilar version of rituximab developed by Celltrion), a biosimilar version of rituximab developed by Biocad, Ublituximab (LFB-R603, a transgenically produced mAb targeting CD20 developed by GTC Biotherapeutics (LFB Biotechnologies)), PF-05280586 (presumed to be a biosimilar version of rituximab developed by Pfizer), Lymphomun (Bi-20, a trifunctional anti-CD20 and anti-CD3 antibody, developed by Trion Pharma), a biosimilar version of rituximab developed by Natco Pharma, a biosimilar version of rituximab developed by iBio, a biosimilar version of rituximab developed by Gedeon Richter/Stada, a biosimilar version of rituximab developed by Curaxys, a biosimilar version of rituximab developed by Coherus Biosciences/Daiichi Sankyo, a biosimilar version of rituximab developed by BioXpress, BT-D004 (a biosimilar version of rituximab developed by Protheon), AP-052 (a biosimilar version of rituximab developed by Aprogen), a biosimilar version of ofatumumab developed by BioXpress, MG-1106 (a biosimilar version of rituximab developed by Green Cross), IBI-301 (a humanized monoclonal antibody against CD20 developed by Innovent Biologics), BVX-20 (a humanized mAb against the CD20 developed by Vaccinex), 20-C2-2b (a bispecific mAb-IFNalpha that targets CD20 and human leukocyte antigen-DR developed by Immunomedics), MEDI-552 (developed (HLA-DR) by MedImmune/AstraZeneca), the anti-CD20/streptavidin conjugates developed by NeoRx (now Poniard Pharmaceuticals), the 2nd generation anti-CD20 human antibodies developed by Favrille (now MMRGlobal), TRU-015, an antibody specific for CD20 fragment developed by Trubion/Emergent BioSolutions, as well as other precloinical approaches by various companies and entities. All aforementioned publications, references, patents and patent applications are incorporated by reference in their entireties. All antibodies disclosed in therein may be used within the present invention.

In some embodiments, the antibody specific for CD20 is selected from the group consisting of rituximab, ocrelizumab, obinutuzumab, ofatumumab, ibritumomab tiuxetan, tositumomab, and ublituximab.

In some embodiments, the antibody specific for CD20 is Rituximab that comprises an HCDR1 region of sequence SYNMH (SEQ ID NO:1), an HCDR2 region of sequence SEQ ID NO:2 (AIYPGNGDTSYNQKFKG), an HCDR3 region of sequence SEQ ID NO:3 (STYYGGDWYFNV), an LCDRI region of sequence SEQ ID NO:4 (RASSSVSYIH), an LCDR2 region of sequence SEQ ID NO:5 (ATSNLAS), and an LCDR3 region of sequence SEQ ID NO:6 (QQWTSNPPT). Rituximab comprises a heavy chain of the sequence SEQ ID NO:7 and a light chain of the sequence of SEQ ID NO:8.

>Heavy chain of Rituximab
SEQ ID NO: 7
QVQLQQPGAELVKPGASVKMSCKASGYTFTSYNMHWVKQTPGRGLEWIG
AIYPGNGDTSYNQKFKGKATLTADKSSSTAYMQLSSLTSEDSAVYYCAR
STYYGGDWYFNVWGAGTTVTVSAASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKAEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK
>Light chain of Rituximab
SEQ ID NO: 8
QIVLSQSPAILSASPGEKVTMTCRASSSVSYIHWFQQKPGSSPKPWIYA
TSNLASGVPVRFSGSGSGTSYSLTISRVEAEDAATYYCQQWTSNPPTFG
GGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

In some embodiments, the antibody specific for CD20 is an antibody which cross-competes with rituximab.

As used herein, the term “cross-competes” refers to single domain antibodies which share the ability to bind to a specific region of an antigen. In the present disclosure the single domain antibody that “cross-competes” has the ability to interfere with the binding of rituximab in a standard competitive binding assay. Such a single domain antibody may, according to non-limiting theory, bind to the same or a related or nearby (e.g., a structurally similar or spatially proximal) epitope as the single domain antibody with which it competes, i.e rituximab. Cross-competition is present if single domain antibody A reduces binding of single domain antibody B at least by 60%, specifically at least by 70% and more specifically at least by 80% and vice versa in comparison to the positive control which lacks one of said single domain antibodies.

As the skilled artisan appreciates competition may be assessed in different assay set-ups. One suitable assay involves the use of the Biacore technology (e.g., by using the BIAcore 3000 instrument (Biacore, Uppsala, Sweden)), which can measure the extent of interactions using surface plasmon resonance technology. Another assay for measuring cross-competition uses an ELISA-based approach. Furthermore, a high throughput process for “binning” antibodies based upon their cross-competition is described in International Patent Application No. WO2003/48731.

According to the present invention, the cross-competing antibody as above described retain the activity of the rituximab.

Typically the active ingredient of the present invention (e.g. depleting agent of the present invention) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1. Differential gene expression between rheumatic heart disease (RHD) valves and control valves (CTRL). A, Exploratory volcano plot analysis in cohort (n=23): Out of 758 experimental genes analyzed, many genes are upregulated. MS4A1 and MS4A2 looks of interest. B, Gene set expression in cohort (n=23): Box plots demonstrate differences in gene expression for CD20-gene related (MS4A1, MS4A2).

### EXAMPLE

Rheumatic heart disease (RHD) ranks as one of the most serious cardiovascular scourges of the past century and remains a force to be reckoned with in the developing world¹.

The nCounter® NanoString® technology is a multiplex analysis system. It is currently the only system capable of quantifying up to 800 nucleic acids of formalin-fixed paraffin-embedded (FFPE) tissue with one single reaction and without amplification step². Avoiding amplification step allow to get rid of typical analysis bias (i.e cross-hybridization, background noise, level of detection . . . ). Despite this difference with others technologies, nCounterR system has been demonstrated to show comparable results between FFPE and fresh frozen samples and in that a sensitivity that is higher than that of microarrays and about equal to that of RT-PCR^{2,3}. Beyond the advantage of being performed on the same sample used for light microscopy, nCounterR offer the opportunity for analysis on large retrospective and longitudinal analyses of archived samples in the setting of decentralized multicenter studies. We have taken advantage of these different characteristics to analyze transcriptomic profiles from FFPE valves of RHD patients.

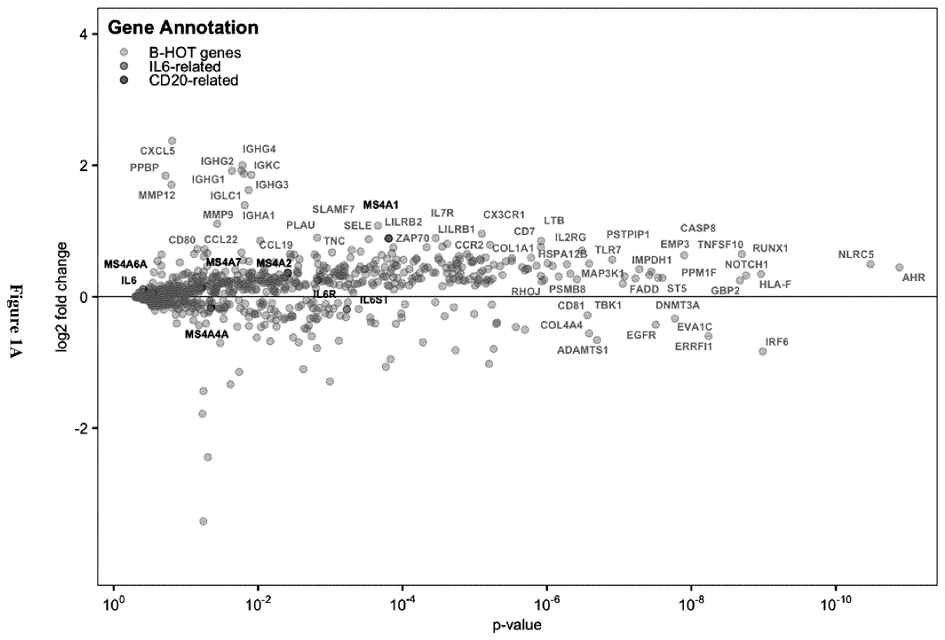

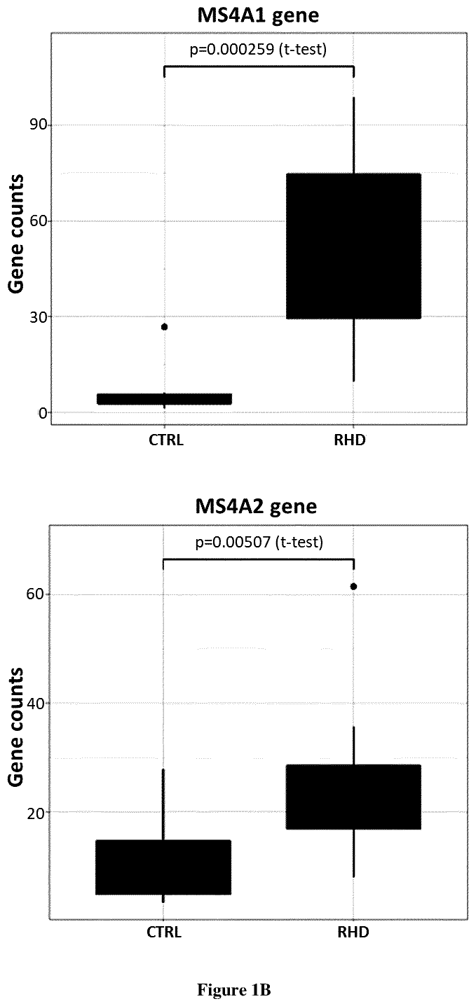

Using the B-HOT panel, developed in part by our team⁴, and based on heatmap analysis (data not shown), our first results show two distinct transcriptomic profiles between inflammatory burden region of RHD valves patients (n=13) and control valves (without lesions, n=10). Our differential gene expression analysis between those 2 groups show several genes overexpressed in RHD group compared to control valves (FIG. 1A). Interestingly, and as potential therapeutic targets, genes related to CD20 (MS4A1, MS4A2) are among genes that are differentially expressed. Box plots gene expression specific to those genes give insights on a strong tendency for an overexpression of CD20-genes related (FIG. 1B).

Taken together, these results suggest a potential role for CD20 in inflammatory responses of RHD valves lesions. Rituximab (CD20) would be potential therapeutic molecules to be used on RHD patients.

### REFERENCES

Throughout this application, various references describe the state of the art to which this invention pertains. The disclosures of these references are hereby incorporated by reference into the present disclosure.
1. Marijon, E., Celermajer, D. S. & Jouven, X. Rheumatic Heart Disease—An Iceberg in Tropical Waters. http: dx.doi.org.proxy.insermbiblio.inist.fr 10.1056 NEJMe1705840 https://www-nejm-org.proxy.insermbiblio.inist.fr/doi/10.1056/NEJMe1705840 (2017) doi:10.1056/NEJMe1705840.
2. Geiss, G. K. et al. Direct multiplexed measurement of gene expression with color-coded probe pairs. Nat. Biotechnol. 26, 317-325 (2008).
3. Payton, J. E. et al. High throughput digital quantification of mRNA abundance in primary human acute myeloid leukemia samples. J. Clin. Invest. 119, 1714-1726 (2009).
4. Mengel, M. et al. Banff 2019 Meeting Report: Molecular diagnostics in solid organ transplantation-Consensus for the Banff Human Organ Transplant (B-HOT) gene panel and open source multicenter validation. Am. J. Transplant. 20, 2305-2317 (2020).

1. A method of treating a rheumatic heart disease in a patient in need thereof comprising administering to the patient a therapeutically effective amount of a B-cell depleting agent.

2. The method of claim 1 wherein the B cell depleting agent is an antibody having specificity for CD20.

3. The method of claim 2 wherein the antibody specific for CD20 is selected from the group consisting of rituximab, ocrelizumab, obinutuzumab, ofatumumab, ibritumomab tiuxetan, tositumomab, and ublituximab.

4. The method of claim 2 wherein the antibody specific for CD20 is rituximab.
